(19) United States
(12) Patent Application Publication
(10) Pub. No.: US 2011/0213422 A1
(43) Pub. Date: Sep. 1, 2011

(54) ORTHOPEDIC FIXATION ASSEMBLY AND METHOD OF USE

(76) Inventors: Jamy Gannoe, West Milford, NJ (US); Brian Adams, Iowa City, IA (US); Tara Ziolo, Hewitt, NJ (US); Jeff Tyber, Bethlehem, PA (US)

(21) Appl. No.: 12/887,037

(22) Filed: Sep. 21, 2010

Related U.S. Application Data
(60) Provisional application No. 61/277,212, filed on Sep. 22, 2009.
(60) Provisional application No. 61/340,431, filed on Mar. 17, 2010.

(51) Int. Cl. A61B 17/84

(52) U.S. Cl. 606/300

(57) ABSTRACT

An orthopedic fixation assembly for compressing bones includes a bone anchor coupled to a washer. The bone anchor includes a shaft, where the shaft extends along a radius of curvature. The bone anchor also includes a threaded end for anchoring into bone or bone fragments. The washer includes a body and a bore extending therethrough. The washer is coupled to the bone anchor by receiving the shaft within the bore. The washer is compressed against a surface of the underlying bone causing the washer to deform and apply a compressive force on the wire body and on the bone fragments.

### CROSS-REFERENCE TO RELATED APPLICATIONS

This non-provisional application claims the benefit of provisional application No. 61/277,212, filed Sep. 22, 2009, and provisional application No. 61/340,431, filed Mar. 17, 2010, the entire contents of both are herein incorporated by reference.

### FIELD OF THE INVENTION

This invention relates to the field of orthopedic implant devices, and, more particularly, to an orthopedic fixation assembly for providing a compressive force and interfragmentary fixation to secure two or more bone fragments or bones together.

### BACKGROUND OF THE INVENTION

Orthopedic implant devices are often used to repair or reconstruct bones and joints due to bone fractures, degenerative bone conditions, and other similar types of injuries. Frequently, these orthopedic devices require that bone fragments, due to bone fractures or bones that are cut during a surgical operation (i.e., an osteotomy), must be kept together for long periods of time or short periods intraoperatively under a sustained force across the fracture site in order to promote healing and for stabilization. As such, these orthopedic implant devices have several functions. These devices may be used to realign bone segments, to apply interfragmental compression to bone fragments, or to restore native geometry.

Interfragmental compression is the ideal condition for bone healing, as stated by Wolff's law: bone grows under load and resorbs (i.e., is removed) in the absence of loads. To achieve interfragmental compression, a screw, a plate or multiple crossed wires are used. At times, there is not enough space for a screw, and multiple wires do not apply the required compression needed to hold and compress the bone fragments.

There is, therefore, a need for an orthopedic implant device assembly and method of use that overcomes some or all of the previously delineated drawbacks of prior orthopedic implant device assemblies.

### SUMMARY OF THE INVENTION

An object of the invention is to overcome these and other drawbacks of previous inventions.

Another object of the invention is to provide a novel and useful orthopedic fixation assembly that may be utilized to secure multiple bones fragments or bones together.

Another object of the invention is to provide an orthopedic fixation assembly that may be utilized to secure the implant bone interface.

Another object of the invention is to provide an orthopedic fixation assembly for converting temporary or provisional fixation to permanent fixation assembly.

Another object of the invention is to provide an orthopedic fixation assembly for holding and compressing a bone where screw access is not possible.

In a first non-limiting aspect of the invention, an orthopedic fixation assembly for compressing bones is provided and includes a bone anchor and a compression washer. The bone anchor includes a shaft, where the shaft extends along a radius of curvature. The compression washer includes a body and a bore extending through the body. The bore extends through the body through a bore axis. The shaft is adapted to be inserted into the bore.

In a second non-limiting aspect of the invention, an orthopedic fixation assembly is provided including a first member, where the first member has a wire body having distal and proximal ends, where the distal end is threaded and is inserted across or through a bone. The fixation assembly also includes a second member having a circular shaped body including a slot and a through aperture, where the wire body is received in the through aperture. The circular shaped body, when positioned on the wire body, and pressed against a proximal fragment, causes the circular shaped slotted body to deform, with the result being a compressive force on the wire body and on the bone fragments, and a coupling of the second member to the wire body.

In a third non-limiting aspect of the invention, a method for inserting an orthopedic fixation assembly into bone fragments is provided and includes several non-limiting steps. In one step, a Kirschner wire is provided having a first threaded end and a second end. The Kirschner wire is provided with a compression washer. In another step, the first end is coupled to a drill and the threaded end is inserted into bone fragments. The Kirschner wire is drilled into bone fragments until the threaded end of Kirschner wire anchors into a bone. In another step, the compression washer is forced down the Kirschner wire until it makes contact with the exterior surface of a bone. In one embodiment, the compression washer may receive more force to deform the washer against the exterior surface of the bone. The compression washer is securely coupled to the Kirschner wire through friction generated by the compression washer via deformation of the compression washer. This results in a compressive force on the bone fragments.

In a fourth non-limiting aspect of the invention, an orthopedic fixation assembly is provided including a bone anchor coupled to a compression washer. The bone anchor includes a wire-shaped body with distal and proximal ends, with the distal end being threaded and inserted across or through a bone or bones. The compression washer includes a generally annular body having a slot and a through-aperture, with the wire body being received within the through-aperture. The bone anchor is inserted into bone or bones and the compression washer is positioned onto the proximal end of bone anchor and forced down the wire body against exterior bone to generate a compressive against the bone or bones. The compression washer may be crushed (or crimped), in this compressive position, to create an interference on the wire to maintain the compressive force.

### BRIEF DESCRIPTION OF THE DRAWINGS

A further understanding of the invention can be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of an orthopedic fixation assembly inserted into the distal phalanx, proximal phalanx, and first metatarsal bones of a patient's foot according to the preferred embodiment of the invention;

FIG. 2A is a perspective view of the compression washer used in the orthopedic fixation assembly, which was shown in FIG. 1;

FIG. 2B is a perspective cross-sectional view of the compression washer, which was shown in FIGS. 1 and 2A of the preferred embodiment;

FIG. 3A is a front view of the Kirschner wire used in the orthopedic fixation assembly, which was shown in FIG. 1;

FIG. 3B is a partial front view of a threaded end of the Kirschner wire, which was shown in FIG. 3A;

FIG. 4A is a perspective view of a compression washer according to an alternate embodiment of the invention;

FIG. 4B is a perspective cross-sectional view of the compression washer, which was shown in FIG. 4A according to an alternate embodiment of the invention;

FIG. 5 is a perspective view of the assembled orthopedic fixation assembly inserted into the distal phalanx, middle phalanx, and the proximal phalanx bones of a patient's hand according to another embodiment of the invention;

FIG. 6 is a flow chart illustrating the method of coupling the orthopedic fixation assembly shown in FIGS. 2A-3B to the distal phalanx, the middle phalanx and the proximal phalanx bones in a patient's hand according to an embodiment of the invention;

FIG. 7 is a perspective view of a claw-disc compression washer according to an alternate embodiment of the invention;

FIG. 8 is a perspective view of a compression washer shown in FIG. 2A, however including suture holes according to an alternate embodiment of the invention;

FIG. 9 is a perspective view of a compression washer according to another alternate embodiment of the invention;

FIG. 10 is a perspective view of the compression washer shown in FIG. 9, however including an aperture adapted to receive a screw according to another alternate embodiment of the invention;

FIG. 11 is a perspective view of a compression washer according to an alternate embodiment of the invention;

FIG. 12 is a perspective cross-sectional view of the compression washer shown in FIG. 11 but without a slot, in accordance with an alternate embodiment of the invention.

FIG. 13 is a perspective view of a crushable compression washer according to an alternate embodiment of the invention;

FIG. 14A is a perspective view of a collet-style compression washer assembly according to an alternate embodiment of the invention;

FIG. 14B is a perspective cross-sectional view of a collet member shown in FIG. 14A according to an alternate embodiment of the invention;

FIG. 14C is a perspective cross-sectional view of a bushing member shown in FIG. 14A according to an alternate embodiment of the invention;

FIG. 14D is a perspective cross-sectional view of the compression washer assembly shown in FIG. 14A according to an alternate embodiment of the invention; and

FIG. 15A is another perspective view of compression washer assembly shown in FIGS. 14A-14D, but inserted into the second proximal phalanx of a patient's hand according to an alternate embodiment of the invention.

FIG. 15B is another perspective view of the compression washer assembly shown in FIGS. 14A-14D inserted into the second proximal phalanx of a patient's hand according to an alternate embodiment of the invention.

FIG. 15C is yet another perspective view of the compression washer assembly shown in FIGS. 14A-14D inserted into the second proximal phalanx of a patient's hand according to an alternate embodiment of the invention.

FIG. 16 is a flow chart illustrating the method of coupling the compression washer assembly shown in FIGS. 14A-14D to the second proximal phalanx bone in a patient's hand according to an alternate embodiment of the invention.

### DETAILED DESCRIPTION OF THE INVENTION

The invention may be understood more readily by reference to the following detailed description of preferred embodiment of the invention. However, techniques, systems, and operating structures in accordance with the invention may be embodied in a wide variety of forms and modes, some of which may be quite different from those in the disclosed embodiment.

Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

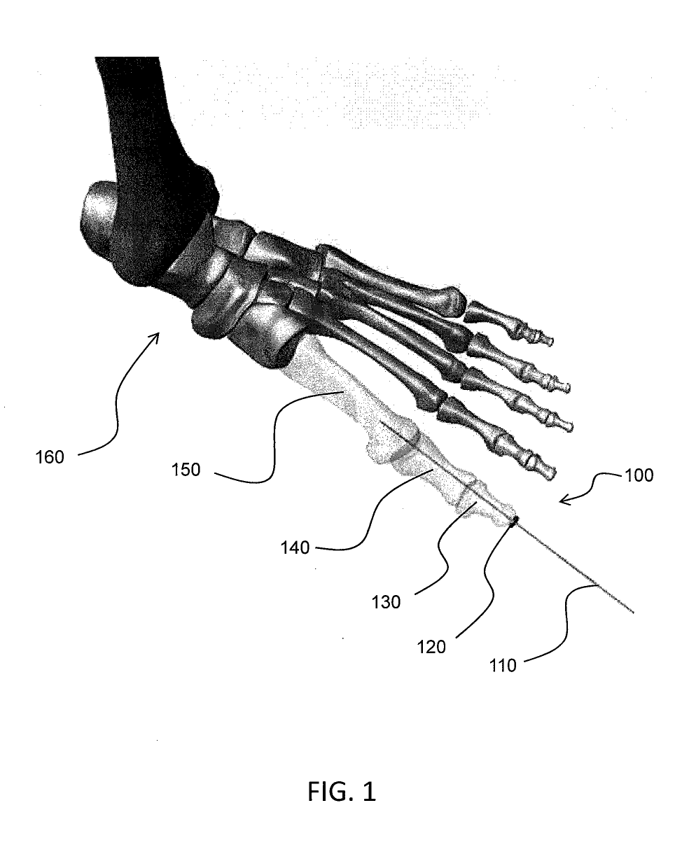

Referring now to FIG. 1, there is shown an orthopedic fixation assembly 100, which is made in accordance with the teachings of the preferred embodiment of the invention. As shown, orthopedic fixation assembly 100 includes a generally cylindrical Kirschner wire 110 slideably coupled to a generally circular compression washer 120, which will be shown and described below. The orthopedic fixation assembly 100 is provided to be inserted across any bone or through a plurality of bones such as, in one non-limiting example, the metatarsal bone 150, the proximal phalanx bone 140, and the distal phalanx bone 130 in the foot 160, although in other embodiments, the orthopedic fixation assembly 100 is provided to be inserted into substantially any other bones or parts of bones and on ligaments or tendons. The orthopedic fixation assembly 100 may be utilized for providing compression to bones where access to these bones prevents screws from being utilized. It should be appreciated that orthopedic fixation assembly 100 is connected across bones or to bones in order to compress bones or bone fragments or to hold them in place to promote healing using the Kirschner wire 110 to apply compression and interfragmentary fixation. The orthopedic fixation assembly 100 is convertible from temporary or provisional fixation to more permanent fixation without additional steps.

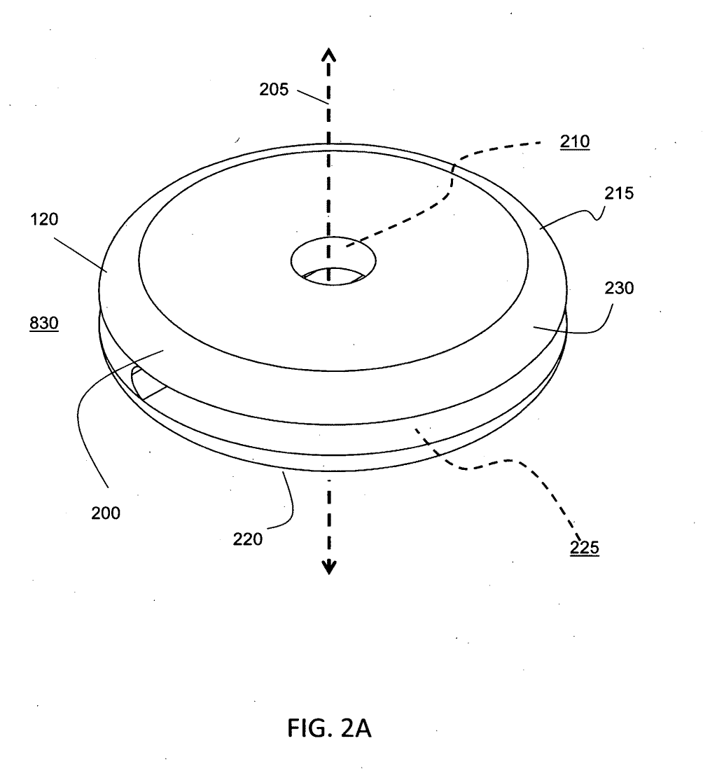

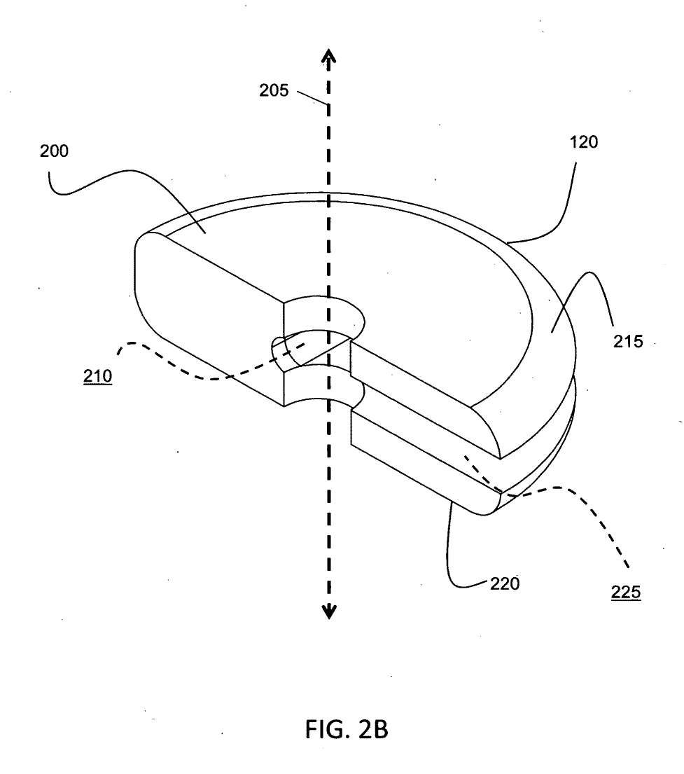

Also, as shown in FIGS. 2A and 2B, compression washer 120 has a generally circular disc-shaped body 200 and includes a hole 210, which may be at an angle to the vertical axis 205 in order to achieve the friction required to hold the body 200 onto the Kirschner wire 110 and apply compression. The hole 210 traverses through body 200 and extends from a first surface 215 and emanates from an opposed surface 220 (i.e., hole 210 is at an angle with surfaces 215 and 220). The diameter of hole 210 may be machined to be larger or smaller than the diameter of a complementary Kirschner wire that it receives, such as Kirschner wire 110. Also, compression washer 120 includes a groove or slot 225 partially formed in body 200 along a plane parallel to surfaces 215 and 220, although in other non-limiting embodiments, the compression washer 120 does not include the slot 225. Also, the groove 225 extends into body 200 at a depth that may extend past the vertical axis 205 forming a top disc 230 and a bottom disc 235. The hole 210 receives Kirschner wire 110 (not shown) and cooperates with the groove 225 to apply a controlled compressive force on bones (not shown) affixed with the orthopedic fixation assembly 100 through the movement between the top disc 230 and bottom disc 235. Once the compressive force is applied to the bones by compressing the washer 120, the washer firmly holds its place along the Kirschner wire 110. In the embodiment that does not include the slot 225, friction between the compression washer 120 and the Kirschner wire 110 may be provided through other means. In one non-limiting embodiment, the compression washer 120 has a low profile having a diameter of 6 mm or less and a thickness of 2 mm or less, although any other dimensions may be utilized. It should also be appreciated that, in one non-limiting embodiment, the compression washer 120 may be made from PEEK material, although, in other non-limiting embodiments, compression washer 120 may be made from Ultra-high-molecular-weight polyethylene (“UHMWPE”), shape memory metal and polymers that may be activated to apply compression, such as NiTiNOL, implantable materials, or other similar types of materials. The compression washer 120 applies a compressive force on Kirschner wire 110 by frictionally engaging Kirschner wire 110. This friction occurs due to a slight angle change of hole 210 in the body 200 which causes the holes to deform which in turn causes friction between the body 200 and the Kirschner wire 110. This occurs through the offsetting angles or position of the two surfaces 215 and 220 causing locking through friction. This friction is sufficient for maintaining offset position for long periods of time. The compression washer 120 may have a radiolucent or radiopaque body 200 and may be completely implantable inside the human body.

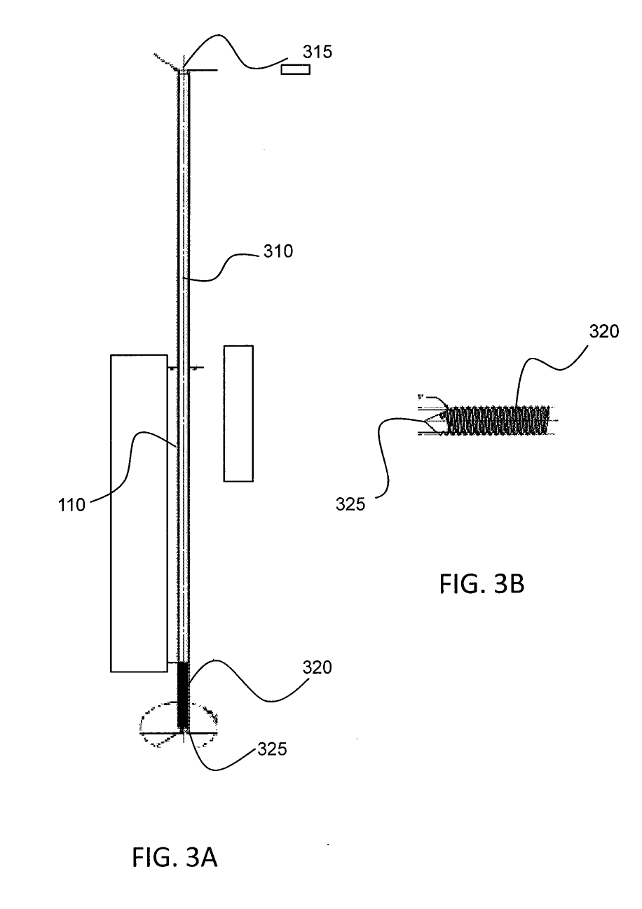

Referring now to FIG. 3A, Kirschner wire 110 includes an elongated body portion 310 having uniform thickness. The body portion 310 may be aligned along its longitudinal axis (such as having a radius that is infinite (such as a straight portion 310 for a very small radius of curvature), or, alternatively, body portion 310 has a radius of curvature for a radius that is between 0.0001 to infinity. The body portion 310 includes a first end 315 and an opposed ringed or threaded portion 320. The first end 315 may be blunt, sharp, flat, or any other shape. Threaded portion 320, as shown in FIG. 3B, has a plurality of circumferential rings or threads on the external surface of threaded portion 320, and terminates into a second end 325 that may be any shape that allows for penetration into bone. In one non-limiting embodiment, second end 325 may have a trocar tip, which may also be threaded. The threaded portion 320 is provided to be inserted into bone or bone fragments (not shown) in order to pierce the cortex of the bone and threadably couple Kirschner wire 110 to bone fragments thereby compressing bones or bone fragments to promote healing using the Kirschner wire 110 and compression washer 120. The threaded portion 320 also provides additional purchase into the bone and prevents the Kirschner wire 110 from backing out of the bone fragments. It should be appreciated that the Kirschner wire 110 may be inserted through an incision or percutaneously. It should also be appreciated that, in one non-limiting embodiment, Kirschner wire 110 may be made from stainless steel (“SST”), although, in other non-limiting embodiments, Kirschner wire 110 may be made from NiTi or other similar types of materials.

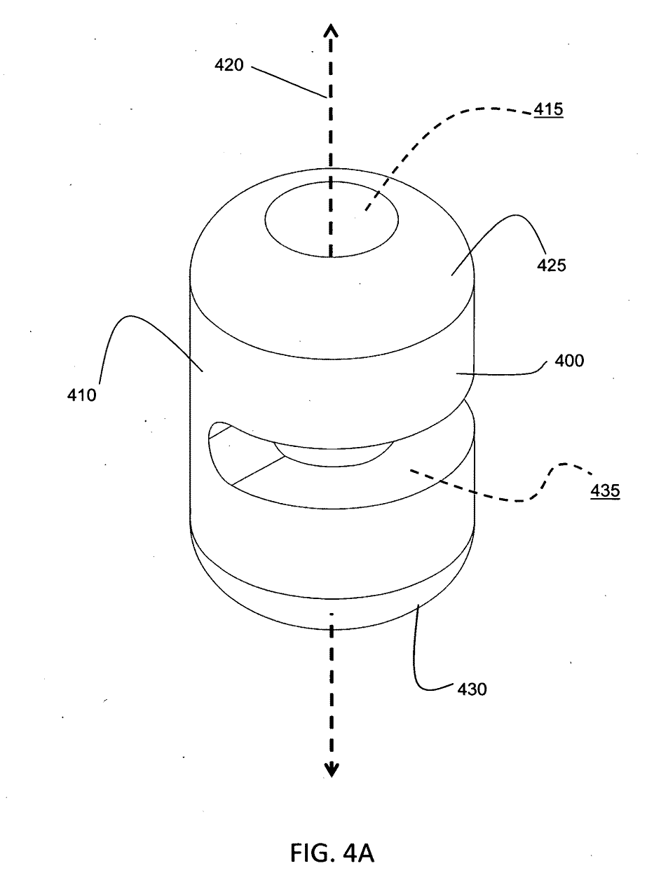

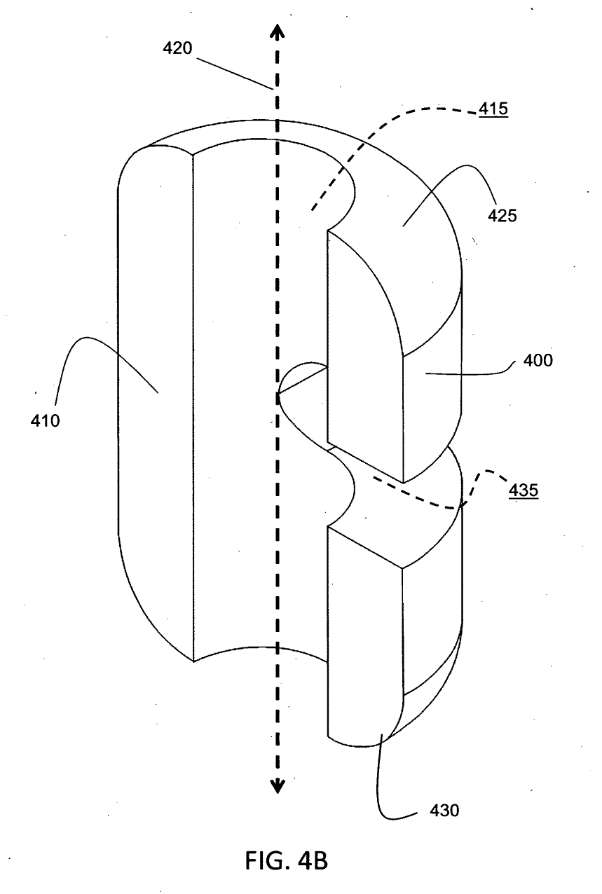

In an alternate, although, non-limiting embodiment, as shown in FIGS. 4A and 4B, orthopedic fixation assembly 100 (not shown) is provided with a generally cylindrical compression washer 400. Compression washer 400, as shown, is substantially similar to compression washer 120 of the embodiment shown and described in FIGS. 2A-2B, and includes a generally cylindrical-shaped body 410. Body 410 includes a hole 415 aligned along vertical axis 420. The hole 415 traverses through body 410 and extends from a first surface 425 and emanates from an opposed surface 430 (i.e., hole 415 is orthogonal to surfaces 425 and 430). Also, compression washer 400 includes a groove 435 partially formed in body 410 in a plane parallel to surfaces 425 and 430. Also, the groove 435 extends into body 410 at a depth that extends past the vertical axis 420. In operation, the hole 415 receives Kirschner wire 110 (not shown) and cooperates with the groove 435 to apply a controlled compressive force on bones (not shown) affixed with the compression washer 400. It should be appreciated that compression washer 400 may be made from Ultra-high-molecular-weight polyethylene (“UHMWPE”), shape memory metal and polymers that may be activated to apply compression, such as NiTiNOL, implantable materials, or other similar types of materials.

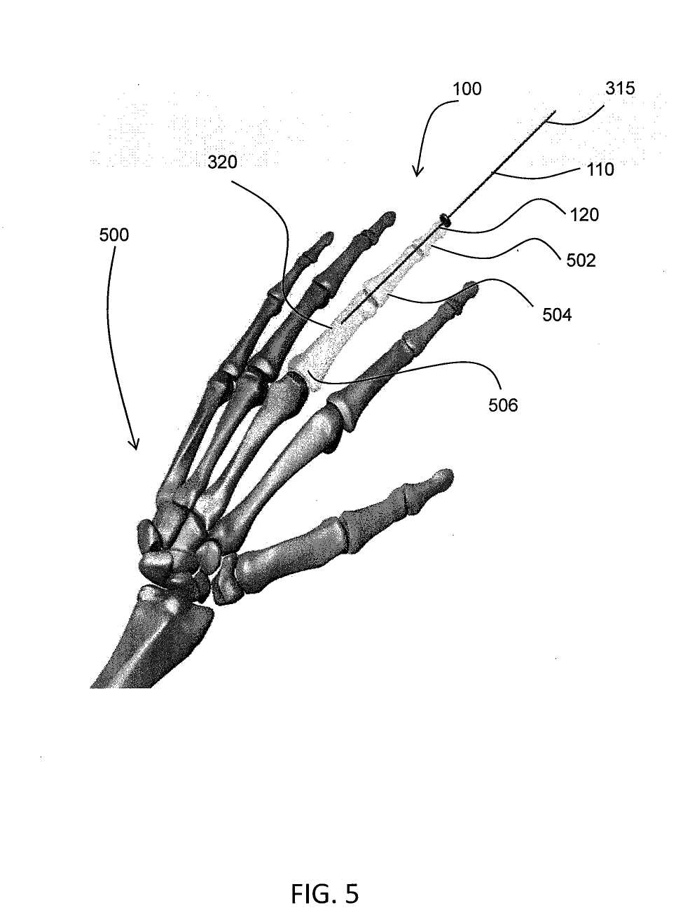

In operation, and as best shown in FIGS. 2A-2B, 3A-3B, 5 and 6, the orthopedic fixation assembly 100 is utilized to connect and compress damaged or fractured bones in the human hand 500 in order to apply compression and interfragmentary fixation so as to promote healing, although the orthopedic fixation assembly 100 may be utilized on any bone in the human body to couple two or more bones together. In one-non limiting example, and as shown in FIG. 5, the orthopedic fixation assembly 100 is coupled to the third distal phalanx bone 502, third middle phalanx bone 504, and third proximal phalanx bone 506. It should be appreciated that the orthopedic fixation assembly 100 may be used within each of the five rays, with a ray representing a line drawn from each distal phalanx bone to the proximal phalanx bone, or any other bones or bone fragments of the human body.

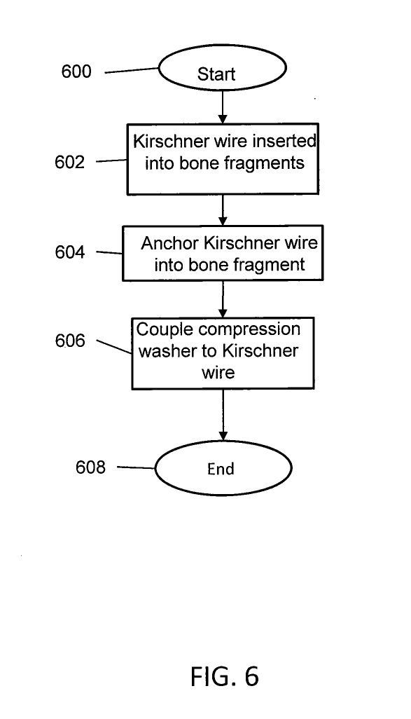

As shown in FIGS. 5 and 6, the orthopedic fixation assembly 110 may be utilized for internal fixation of, in one non-limiting embodiment, bone or bone fragments in the human hand 500. As shown, the method starts in step 600 and proceeds to step 602, whereby a Kirschner wire 110 is inserted into bones 502, 504, and 506. In this step, a Kirschner wire 110 is selected and coupled to a standard drill (not shown) to be inserted into bones 502, 504, and 506 or bone fragments. The Kirschner wire 110 may be assembled with the compression washer 120 prior to packaging with the Kirschner wire 110 being received pre-assembled with the compression washer 120. The Kirschner wire 110, coupled to the compression washer 120, is drilled into bones 502, 504, and 506 until threaded portion 320 anchors into, in one example, the third proximal phalanx 506. Next, in step 604, the compression washer 120 is slid down (i.e. moved down manually) the Kirschner wire 110 until it makes contact with the exterior surface of the third distal phalanx bone 502. The compression washer 120 is securely coupled to the Kirschner wire 110, and applies a frictional force on the Kirschner wire 110 to maintain compression. Friction occurs due to a slight angle change of the hole 210 (FIG. 2A) as the body 200 (shown in FIG. 2A) undergoes deformation, causing the hole 210 (FIG. 2A) to change from being undeformed (i.e., in it original uncompressed state) to being converged (i.e., changes its shape due to compression), which in turn causes friction between the body 200 (FIG. 2A) and the Kirschner wire 110. The method ends in step 606.

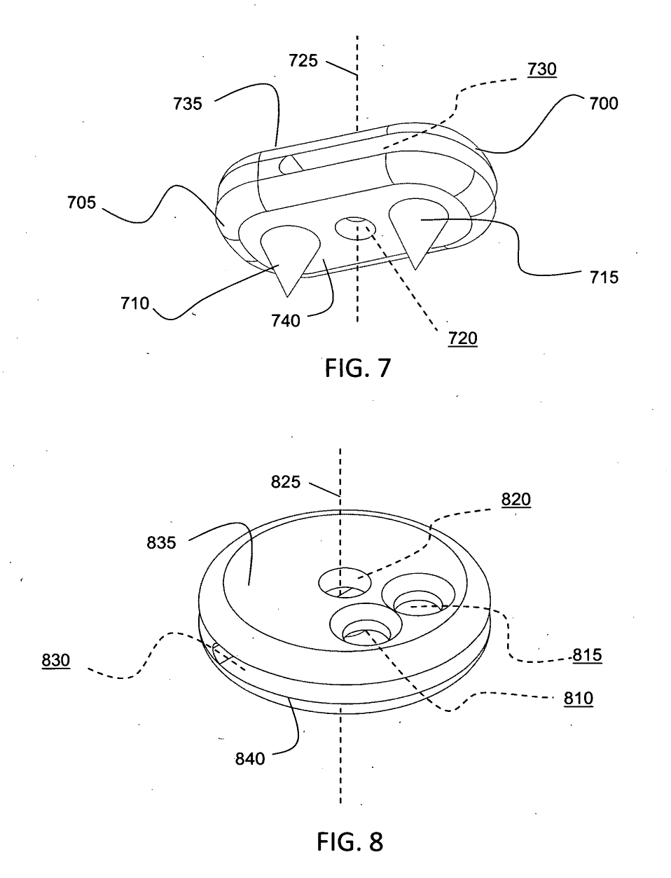

In other alternate but non-limiting embodiments, as shown in FIGS. 7-14D, orthopedic fixation assembly, such as orthopedic fixation assembly 100, may be provided to be utilized with a variety of different compression washers. These compression washers may be made from Ultra-high-molecular-weight polyethylene (“UHMWPE”), shape memory metal and polymers and be activated to apply compression, such as NiTiNOL, implantable materials, or other similar types of materials.

As shown in FIG. 7, claw-disc compression washer 700 has a generally elongated disc shape body 705. Body 705 includes a through hole 720 (i.e., hole 720 travels through body 705) that is aligned along vertical axis 725 and is provided to receive Kirschner wire, such as Kirschner wire 110. Also, compression washer 700 includes a groove 730 partially formed in body 705 in a plane parallel to surfaces 735 and 740. Also, the groove 730 extends into body 705 at a depth that extends past the vertical axis 725. Further, compression washer 700 has a plurality of substantially similar and generally cone-shaped protrusions 710 and 715, which are provided to be inserted into bone fragments (not shown).

As shown in FIG. 8, compression washer 800 is substantially similar to compression washer 120 of the embodiment shown and described in FIGS. 2A-2B, however has a plurality of substantially similar suture holes 810, 815 formed in body 805 that are provided to receive, in one non-limiting embodiment, sutures (not shown). Body 805 includes a through hole 820 aligned along vertical axis 825 and a groove 830 partially formed in body 805 in a plane parallel to surfaces 835, 840. Also, the groove 830 extends into body 805 at a depth that extends past the vertical axis 825.

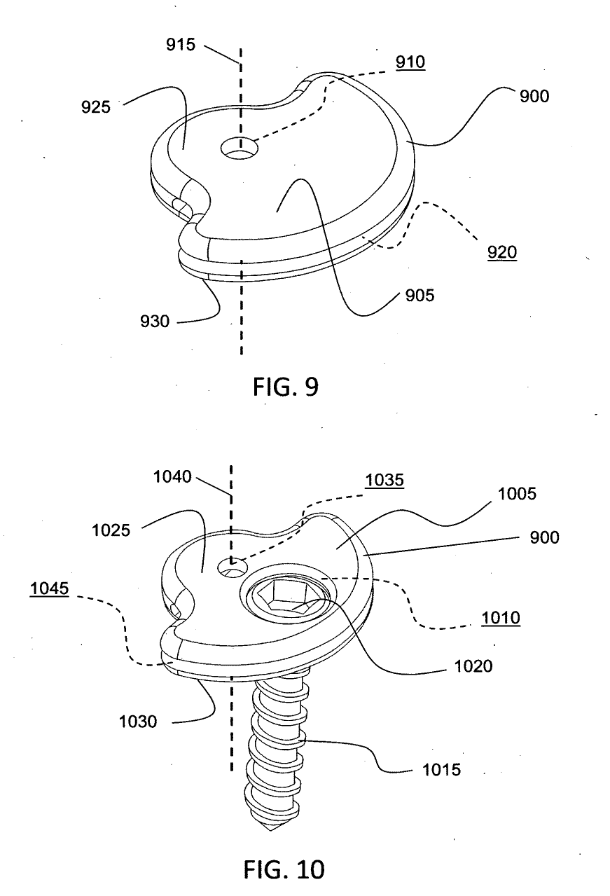

As shown in FIG. 9, compression washer 900 has a generally semi-circular shape body 905, which contains a through hole 910 aligned along vertical axis 915 that is provided to receive a Kirschner wire, such as Kirschner wire 110. Also, compression washer 900 includes a groove 920 partially extending in body 905 in a plane parallel to surfaces 925 and 930, and extends into the body 905 at a depth that extends past the vertical axis 915.

As shown in FIG. 10, compression washer 1000 is substantially similar to compression washer 900 of the embodiment shown and described in FIG. 9, however has a through hole 1010 formed in body 1005 that is provided to receive, in one non-limiting embodiment, a threaded screw 1015. Hole 1010 has a diameter that restricts head 1020 against surface 1025 while allowing the threaded portion of the screw 1015 to emanate from surface 1030. Body 1005 includes hole 1035, aligned along vertical axis 1040, provided to receive, in one example, Kirschner wire 110. Body 1005 also includes a groove 1045 partially formed in body 1005 in a plane parallel to surfaces 1025 and 1030. Also, the groove 1045 extends into body 1005 at a depth that extends past the vertical axis 1040.

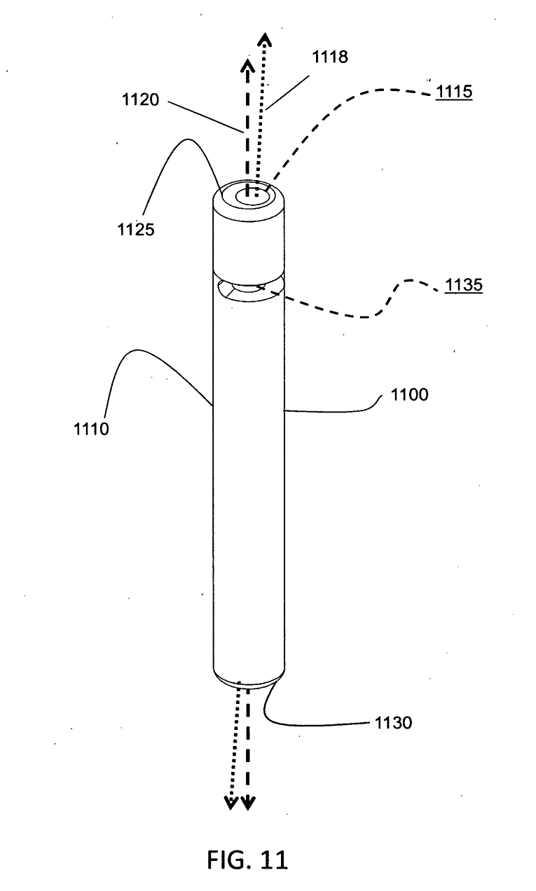

As shown in FIG. 11, orthopedic fixation assembly 100 (not shown) is provided with a generally barrel-shaped compression washer 1100. Compression washer 1100, as shown, is substantially similar to compression washer 120 of the embodiment shown and described in FIGS. 2A-2B, and includes a generally cylindrical-shaped body 1110. Body 1110 includes a hole 1115 that is aligned along axis 1118, which is offset from vertical axis 1120 at an angle in order to achieve the friction required to hold the body 1110 onto the Kirschner wire 110 (shown and described in FIG. 3A). The hole 1115 traverses through body 1110, and extends from a first surface 1125 and emanates from an opposed surface 1130. In one example, the body 1110 is 15 mm in height and 1 mm larger than the diameter of the Kirschner wire 110 (not shown), although the height can vary depending on anatomical or surgical requirements. Also, compression washer 1100 includes a groove 1135 partially formed in body 1110 in a plane parallel to surfaces 1125 and 1130. Also, the groove 1135 extends into body 1110 at a depth that extends past the vertical axis 1120. In operation, the hole 1115 receives Kirschner wire 110 (not shown) and cooperates with the groove 1135 to apply a controlled compressive force on bones (not shown) affixed with the compression washer 1100. It should be appreciated that the compression washer 1100 may be used in all bones where Kirschner wires 110 or screws are currently used. It should also be appreciated that the compression washer 1100 may be used so that the Kirschner wire 110 (not shown) sits flush to the bone while also sticking out of the skin incision in the inserted state. This provides for ease of removal of the Kirschner wire 110 (not shown).

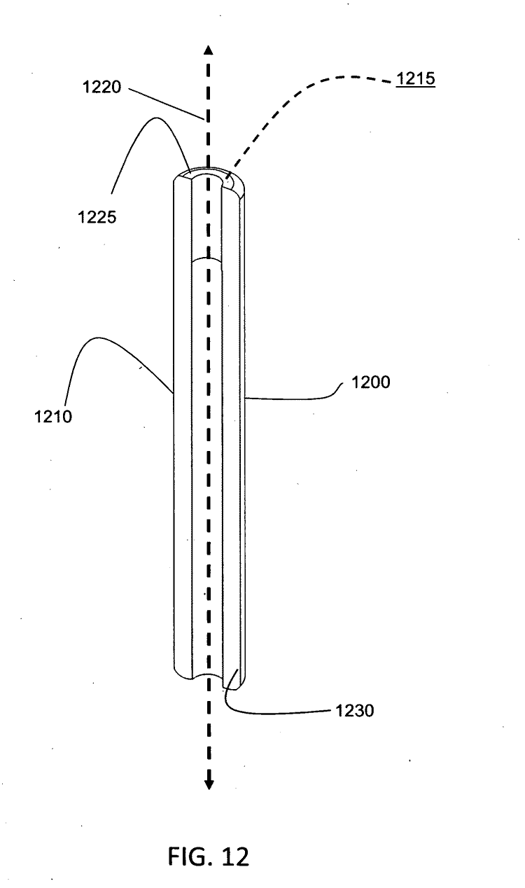

As shown in FIG. 12, orthopedic fixation assembly 100 (not shown) is provided with a generally barrel-shaped compression washer 1200. Compression washer 1200, as shown, is substantially similar to compression washer 120 of the embodiment shown and described in FIGS. 2A-2B, and includes a generally cylindrical-shaped body 1210. Body 1210 includes a hole 1215 that is aligned along axis vertical axis 1220. The hole 1215 provides a friction fit with the Kirschner wire 110 in order to hold the body 1210 onto the Kirschner wire 110 (shown and described in FIG. 3A). The hole 1215 traverses through body 1210, and extends from a first surface 1225 (i.e., penetrates surface 1225) and emanates from an opposed surface 1230. In one example, the body 1210 is 15 mm in height and 1 mm larger than the diameter of the Kirschner wire 110 (not shown), although the height can vary depending on anatomical or surgical requirements. In operation, the hole 1215 receives Kirschner wire 110 (not shown) and provides compression on bones (not shown) affixed with the compression washer 1200 via a friction fit with the Kirschner wire 110. It should be appreciated that the compression washer 1200 may be used in all bones where Kirschner wires 110 or screws are currently used. It should also be appreciated that the compression washer 1200 may be used so that the Kirschner wire 110 (not shown) sits flush to the bone however, also sticks out of the skin incision in the inserted state and provides for ease of removal of the Kirschner wire 110 (not shown).

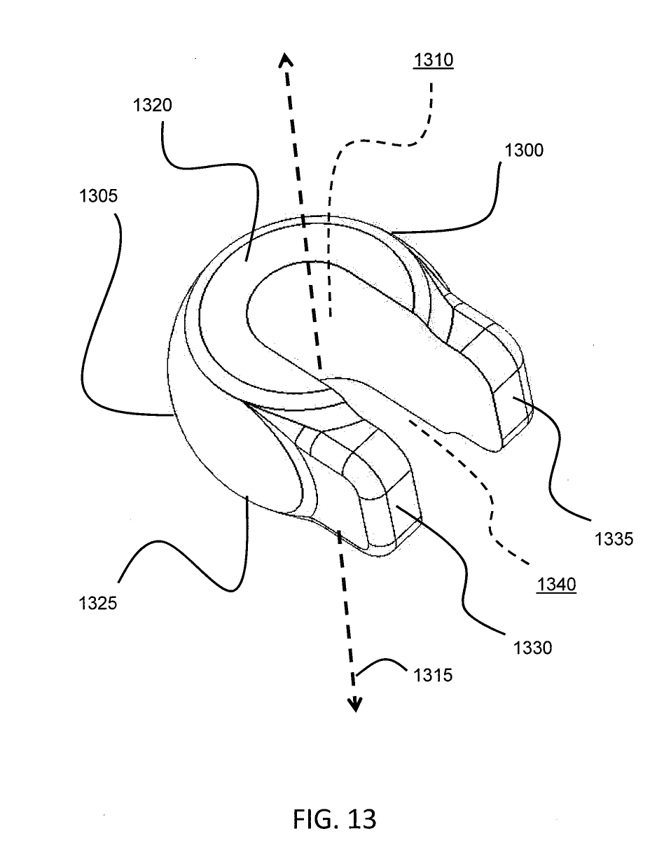

As shown in FIG. 13, orthopedic fixation assembly 100 (not shown) is provided with a compression washer 1300 in the shape of a split bushing to be utilized with Kirschner wire 110 (FIG. 3A) for applying and maintaining compression on bone or bones (not shown). Compression washer 1300, as shown, has a generally annular-shaped body 1305, which may be formed from shape memory metal and polymers, such as NiTiNOL for causing washer 1300 to be crushed (or crimped) on the wire 110. Body 1305 includes a through-aperture 1310 (or bore) traverses body 1305 from first surface 1320 to opposed second surface 1325. The aperture 1310 may pass through the center of body 1305 or, alternatively, may be offset from the center of body 1305. The aperture 1310 is aligned along longitudinal axis 1315, which traverses the body 1305 from first surface 1320 to second surface 1325. Also, aperture 1310 is, preferably, 1 mm larger than the diameter of the Kirschner wire 110 (FIG. 3A), and the height can vary depending on anatomical or surgical requirements. Further, body 1305 includes a plurality of tabs 1330 and 1335 that are separated by a slot 1340, which forms a channel with aperture 1310. The tabs 1330 and 1335 are provided to be deformed or crimped, although in other non-limiting embodiments, the body 1305 may also be deformed or crushed. In operation, the compression washer 1300 receives Kirschner wire 110 (FIG. 3A) within aperture 1310 and applies compression on bones (not shown) via a force applied to the washer against the surface of the bone. With compression being applied, the compression washer 1300 may be crimped or deformed to create an interference fit with the Kirschner wire 110 (FIG. 3A) to maintain the compression.

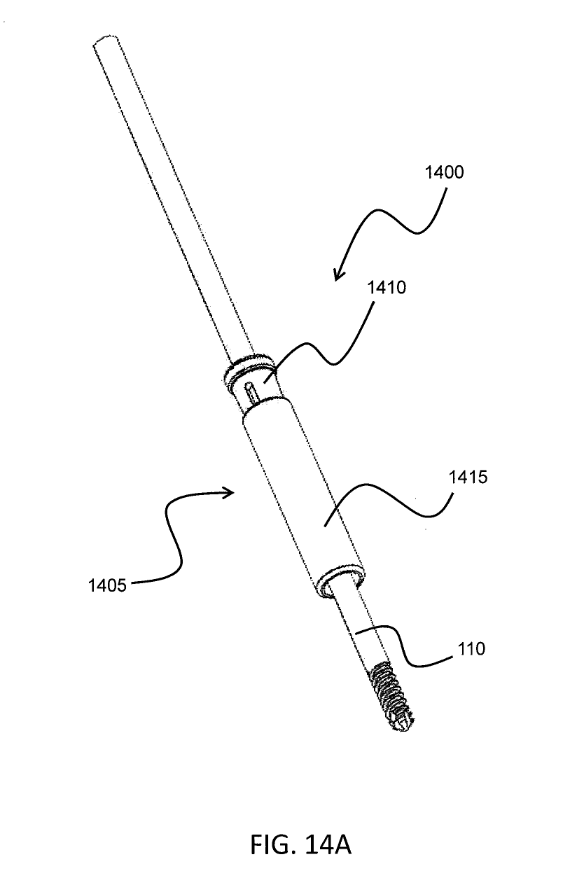

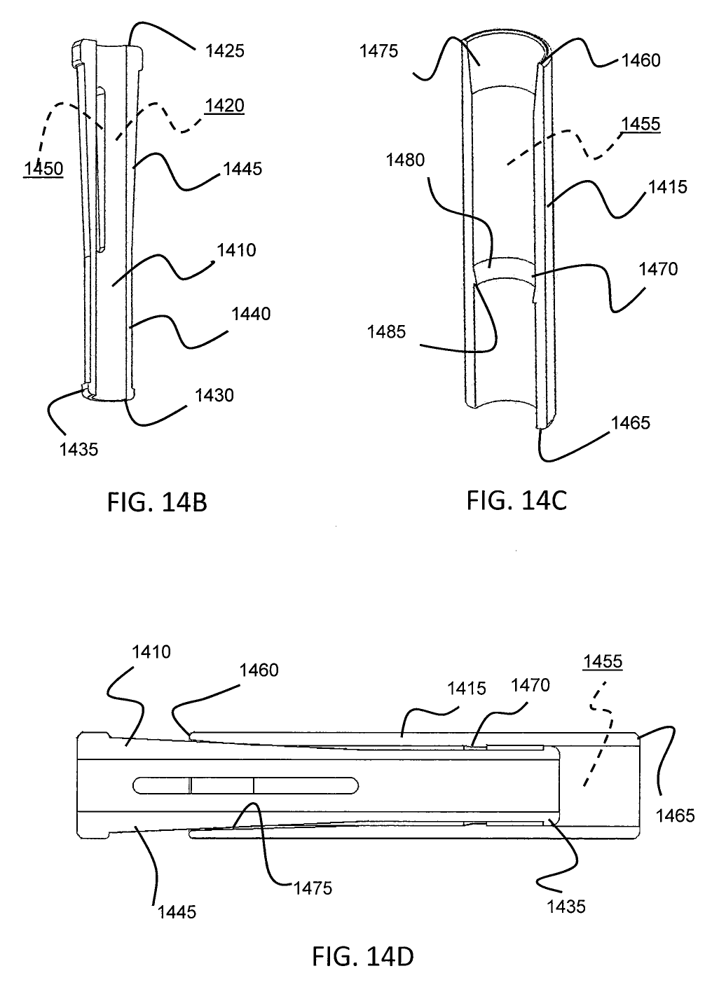

As shown in FIGS. 14A-14D, orthopedic fixation assembly 1400 is provided with a collet and bushing assembly 1405 (hereinafter referred to as “collet assembly 1405”) to be utilized with a Kirschner wire 110 in order to apply and maintain compression on bone or bones (not shown). Collet assembly 1405 includes a generally tubular collet member 1410 for receiving Kirschner wire 110. The collet member 1410 may be made from a shape memory metal and polymers, such as NiTiNOL, and be activated to be deformable, thereby exerting a strong clamping force on Kirschner wire 110 as the collet member 1410 is coupled to Kirschner wire 110 (FIG. 3A), and pulled deeper into bushing member 1415.

As shown in FIG. 14B, collet member 1410 is a collet having a generally cylindrical shape with a central bore or aperture 1420 that longitudinally traverses collet member 1410 from first open end 1425 to second open end 1430. Second open end 1430 includes a protruding edge or lip 1435, which is raised (i.e., protrudes away from the external surface of collet member 1410). The protruding edge 1435 is provided to restrain open end 1430 against the complementary shaped protrusion 1470 (FIG. 14C), and prevent collet member 1410 from sliding out of contact with bushing member 1415 (FIGS. 14A and 14C) once the collet member 1410 is inserted into bushing member 1415 and prior to applying compression. Also, collet member 1410 includes a first tubular portion 1440 having a uniform diameter, which terminates into second tubular portion 1445 having a tapered cross-section (i.e., portion 1445 has an increasing diameter moving from portion 1440 to first end 1425). The diameter of the second tubular portion 1445 becomes larger than the internal diameter of the bushing member 1415 (FIG. 14C) moving from the first tubular portion 1440 to first open end 1425. In addition, the second tubular portion 1445 includes a plurality of elongated slots such as, for example, slot 1450, located circumferentially and equally spaced on the second tubular portion 1445. The first tubular portion 1440 is provided to be received with bushing member 1415 (FIG. 14A), while the second tubular portion 1445 cooperates with the elongated slots, such as elongated slot 1450, to be deformed and cause an interference fit, thereby applying compression against the Kirschner wire 110 (FIG. 3A) and on bone or bones (not shown)

As shown in FIGS. 14B-14D, bushing member 1415 is a bushing having a generally cylindrical shape with a central bore or aperture 1455 that longitudinally traverses bushing member 1415 from first open end 1460 to second open end 1465. Particularly, and as shown in FIG. 14C, bushing member 1415 has a generally smooth exterior surface and a cross-section that includes a raised protrusion 1470 (extending into the central bore 1455) and a slightly tapered surface 1475 at first open end 1460. The protrusion 1470 includes a tapered surface 1480 terminating into an edge 1485 that is orthogonal to the inner surface of the bushing member 1415. The protrusion 1470 (also seen in FIG. 14D) causes edge 1435 to easily slide into the bore 1455, while preventing the collet member 1410 from sliding out of contact with the bushing member 1415 (also seen in FIG. 14D) by providing an obstruction to the protruding edge 1435 (FIG. 14D). Also, tapered surface 1475 is provided to create an interference fit with external surface of second tubular portion 1445 when the collet member 1410 is deformed, thereby preventing collet member 1410 from sliding out of contact with bushing member 1415 when compression is applied. It should be appreciated that, in a fully extended position (i.e., collet member 1410 is loosely coupled to bushing member 1415), the protruding edge 1435 abuts protrusion 1470, and no deformation takes place.

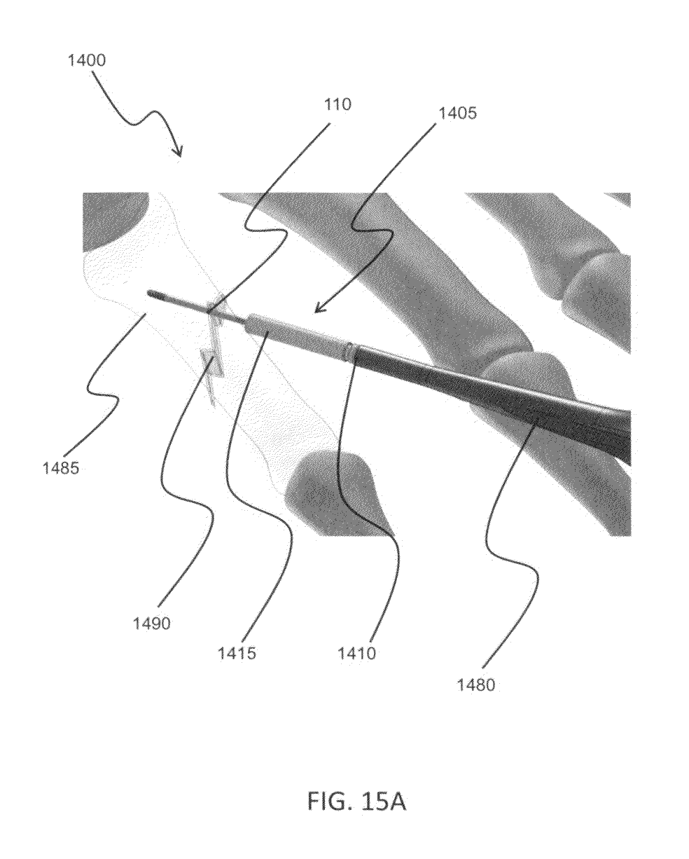

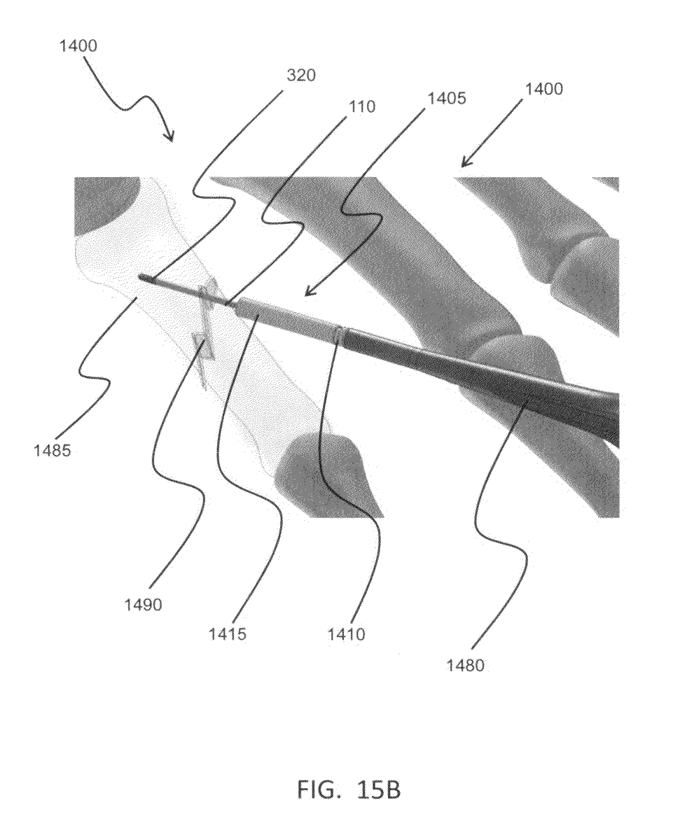

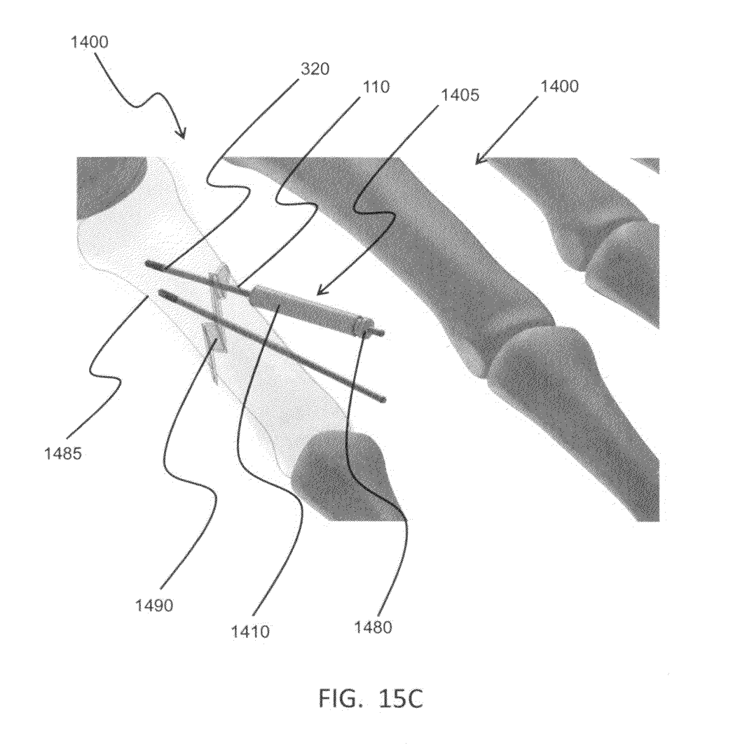

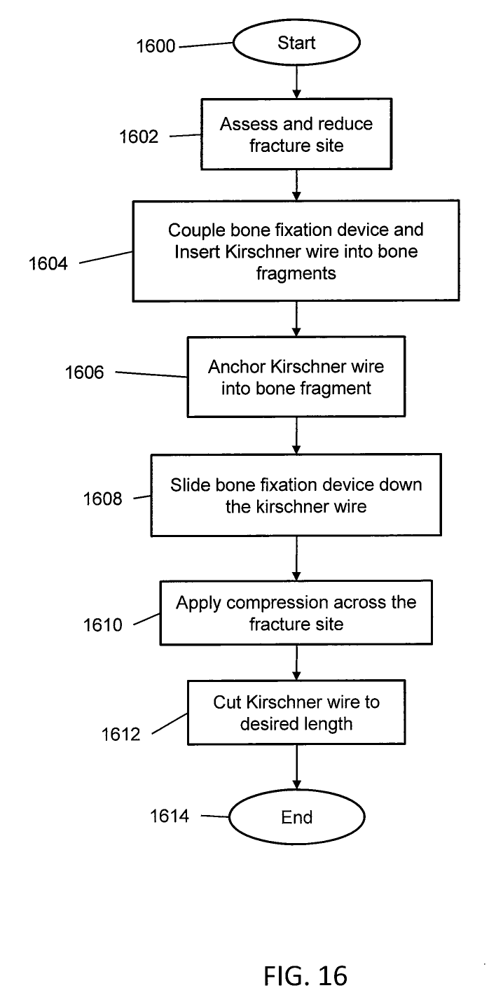

As shown in FIGS. 15A-C and 16, the orthopedic fixation assembly 1400 may be utilized for internal fixation of, in one non-limiting embodiment, bone or bone fragments in the human hand (not shown). As shown, the method starts in step 1600 and proceeds to step 1602, whereby the fracture site 1490, in one example located at the second proximal phalanx bone 1485, is assessed through any conventional means and the fracture site is reduced through conventional methods. Next, in step 1604, a Kirschner wire 110 is coupled to collet assembly 1405 to form the orthopedic fixation assembly 1400. The orthopedic fixation assembly 1400 is inserted into, in one example, the second proximal phalanx bone 1485 to secure the bone fragments. In this step, a Kirschner wire 110 is selected and coupled to a standard wire driver system (not shown) to be inserted into second proximal phalanx bone 1485. The Kirschner wire 110 is assembled with the collet assembly 1405 prior to being coupled to the standard drill wire driver system (not shown) or the Kirschner wire 110 may not be assembled with the collet assembly 1405 prior to being coupled to the standard drill wire driver system (not shown). The Kirschner wire 110 is inserted across a fracture site 1490 into the bone fragments until, in step 1606, the threaded portion 320 (shown in FIG. 3A) anchors into the second proximal phalanx bone 1485. Next, in step 1608, the collet assembly 1405 is either coupled to the Kirschner wire 110 and slid down (i.e. moved manually) by using an accessory pusher 1480 (shown in FIGS. 15A-B) until it makes contact with the exterior surface of the second proximal phalanx bone 1485. In one non-limiting embodiment, if a percutaneous delivery method is used for the Kirschner wire 110, a tiny skin incision is made which is just larger than the diameter of the collet assembly 1405 in order to allow the collet assembly 1405 to pass through skin and make contact with the second proximal phalanx bone 1485. Next in step 1610, as shown in FIG. 15B, the accessory pusher 1480 is forced down against the collet assembly 1405 while pulling up on the Kirschner wire 110 to achieve the desire compression across the bone fragments 1490. Compression is thus obtained by pushing the collet member 1410 into the bushing member 1415. The orthopedic fixation assembly 1400 is able to achieve temporary and sustained compression and can be customized in whatever range is deemed adequate by the surgeon. Friction occurs due to the taper of the collet member 1410 as the portion 1445 (shown in FIG. 14B) undergoes deflection or movement while it is forced into the bushing member 1415. This deflection or movement causes friction between the Kirschner wire 110 and the collet member 1410, with this friction causing the Kirschner wire 110 to lock with the collet assembly 1405. Next, in step 1612, the length of the Kirschner wire 110 is trimmed to the desired length. The method ends in step 1614.

It should be appreciated that a plurality of orthopedic fixation assemblies, such as orthopedic fixation assembly 100, may be inserted into any bones in the human body, in order to heal the bone through compression and interfragmentary compression. It should be appreciated that the orthopedic fixation assembly 100 may be provided as a two-piece assembly comprised of a standard partially threaded Kirschner wire 110 and any of the compression washers shown and described in FIGS. 2A, 4A, and 7-14A.

It should be understood that this invention is not limited to the disclosed features and other similar method and system may be utilized without departing from the spirit and the scope of the invention.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1. An orthopedic fixation assembly for compressing bones comprising:
a bone anchor comprising a shaft; and
a washer comprising a body and a bore extending through the body;
wherein the shaft extends along a radius of curvature;
wherein the bore extends through the body along a bore axis; and
wherein the shaft is adapted to be inserted into the bore.

2. The orthopedic fixation assembly of claim 1, wherein the bore axis is angularly offset from a longitudinal axis extending through the body from a first surface and an opposed second surface.

3. The orthopedic fixation assembly of claim 2, wherein the bore axis is at an acute angle to the longitudinal axis.

4. The orthopedic fixation assembly of claim 1, wherein the shaft comprises a proximal end, a distal end, and a plurality of external threads extending along the shaft from the distal end to the proximal end less than the entirety of the shaft.

5. The orthopedic fixation assembly of claim 4, wherein the plurality of external threads is adapted to be inserted into underlying bone for anchoring the bone anchor to the underlying bone.

6. The orthopedic fixation assembly of claim 1, wherein the shaft further comprises a trocar tip for penetrating underlying bone.

7. The orthopedic fixation assembly of claim 6, wherein the trocar tip is threaded.

8. The orthopedic fixation assembly of claim 6, wherein the trocar tip is non-threaded.

9. The orthopedic fixation assembly of claim 1, wherein the washer further comprises a slot extending through the body for less than an entirety of the body, wherein the slot resides along a plane that is orthogonal to the longitudinal axis.

10. The orthopedic fixation assembly of claim 9, wherein the slot extends beyond the longitudinal axis.

11. The orthopedic fixation assembly of claim 1, wherein the washer further comprises at least one screw hole passing therethrough.

12. The orthopedic fixation assembly of claim 1, wherein the washer further comprises at least one suture hole.

13. The orthopedic fixation assembly of claim 1, wherein the bone anchor cooperates with the washer to apply a compressive force to underlying bones.

14. The orthopedic fixation assembly of claim 13, wherein the compressive force is applied by deforming the bore in relation to the bore axis.

15. The orthopedic fixation assembly of claim 14, wherein the deformed bore causes the washer to create an interference lock with the bone anchor.

16. The orthopedic fixation assembly of claim 1, wherein the washer is disc shaped.

17. The orthopedic fixation assembly of claim 1, wherein the washer is tubular in shape.

18. The orthopedic fixation assembly of claim 1, wherein the washer is a bushing, said bushing being made from a deformable material for shrinking on said bone anchor.

19. The orthopedic fixation assembly of claim 1, wherein said washer is a bushing, said bushing comprising a shape memory material for creating an interference on said bone anchor.

20. A method for applying compression to bones, comprising:
providing an orthopedic fixation assembly comprising a bone anchor and a washer;
wherein the bone anchor comprises a shaft, a threaded end, and a smooth end, wherein the shaft extends along a radius of curvature; and
wherein the washer comprises a body and a bore extending through the body, wherein the body extends through the bore along a bore axis;
coupling the bone anchor to a drill;
inserting the shaft into a plurality of bones to anchor the threaded end inside at least one bone of the plurality of bones;
slideably coupling the bone anchor to the washer by inserting the smooth end into the bore;
sliding the washer along the shaft until the washer contacts an exterior surface of the at least one bone;
deforming the washer against the exterior surface of the at least one bone to apply compression to the plurality of bones.

21. The method of claim 20, wherein the bore axis is angularly offset from a longitudinal axis extending through the body from a first surface and an opposed second surface.

22. The method of claim 21, wherein the bore axis is at an acute angle to the longitudinal axis.

23. The method of claim 20, wherein the shaft comprises a proximal end, a distal end, and a plurality of external threads extending along the shaft from the distal end to the proximal end less than the entirety of the shaft.

24. The method of claim 23, wherein the plurality of external threads is adapted to be inserted into underlying bone for anchoring the bone anchor to the underlying bone.

25. The method of claim 20, wherein the shaft further comprises a trocar tip for penetrating underlying bone.

26. The method of claim 25, wherein the trocar tip is threaded.

27. The method of claim 25, wherein the trocar tip is non-threaded.

28. The method of claim 20, wherein the washer further comprises a slot extending through the body for less than an entirety of the body, wherein the slot resides along a plane that is orthogonal to the longitudinal axis.

29. The method of claim 28, wherein the slot extends beyond the longitudinal axis.

30. The method of claim 20, wherein the washer further comprises at least one screw hole passing therethrough.

31. The method of claim 20, wherein the washer further comprises at least one suture hole.

32. The method of claim 20, wherein the bone anchor cooperates with the washer to apply a compressive force to underlying bones.

33. The method of claim 20, wherein washer applies a compressive force on the bone anchor by deforming the bore in relation to the bore axis.

34. The method of claim 33, wherein the deformed bore causes the washer to create an interference lock with the bone anchor.

35. The method of claim 20, wherein the washer is disc shaped.

36. The method of claim 20, wherein the washer is tubular in shape.

37. The method of claim 20, wherein the washer is a bushing, said bushing being made from a deformable material for shrinking on said bone anchor.

38. The method of claim 20, wherein said washer is a bushing, said bushing comprising a shape memory material for creating an interference on said bone anchor.

39. An orthopedic fixation assembly for applying compression to underlying bone, comprising:
a bone anchor comprising a shaft extending along a radius of curvature; and
a washer comprising a body and a bore extending therethrough;
wherein the shaft comprises a proximal end, a distal end, and a plurality of external threads extending along the shaft from the distal end to the proximal end less than the entirety of the shaft
wherein the body resides within the bore along a bore axis; and
wherein the shaft is adapted to be inserted into the bore.

40. The orthopedic fixation assembly of claim 39, wherein the bore axis is angularly offset from a longitudinal axis extending through the body from a first surface to an opposed second surface.

41. The orthopedic fixation assembly of claim 40, wherein the bore axis is at an acute angle to the longitudinal axis.

42. The orthopedic fixation assembly of claim 39, wherein the plurality of external threads is adapted to be inserted into the underlying bone for anchoring the bone anchor to the underlying bone.

43. The orthopedic fixation assembly of claim 39, wherein the shaft further comprises a trocar tip for penetrating underlying bone.

44. The orthopedic fixation assembly of claim 43, wherein the trocar tip is threaded.

45. The orthopedic fixation assembly of claim 43, wherein the trocar tip is non-threaded.

46. The orthopedic fixation assembly of claim 39, wherein the washer further comprises a slot extending through the body for less than an entirety of the body, wherein the slot resides along a plane that is orthogonal to the longitudinal axis.

47. The orthopedic fixation assembly of claim 46, wherein the slot extends beyond the longitudinal axis.

48. The orthopedic fixation assembly of claim 39, wherein the bone anchor cooperates with the washer to apply a compressive force to the underlying bones.

49. The orthopedic fixation assembly of claim 39′, wherein washer applies a compressive force on the bone anchor by deforming the bore in relation to the bore axis.

50. The orthopedic fixation assembly of claim 49, wherein the deformed bore causes the washer to create an interference lock with the bone anchor.

51. The orthopedic fixation assembly of claim 39, wherein the washer is disc shaped.

52. The orthopedic fixation assembly of claim 39, wherein the washer is tubular in shape.

53. The orthopedic fixation assembly of claim 39, wherein the washer is a bushing, said bushing being made from a deformable material for shrinking on said bone anchor.

54. The orthopedic fixation assembly of claim 39, wherein said washer is a bushing, said bushing comprising a shape memory material for creating an interference on said bone anchor.
